(19) United States
(12) Patent Application Publication
(10) Pub. No.: US 2024/0057896 A1
(43) Pub. Date: Feb. 22, 2024

(54) Waveguide for Optical Sensor

(71) Applicant: APPLE INC, Cupertino, CA (US)

(72) Inventors: Edward Vail, Menlo Park, CA (US); Ehsan Shah-Hosseini, San Francisco, CA (US); Joseph M. Schmitt, Santa Clara, CA (US)

(21) Appl. No.: 18/234,786

(22) Filed: Aug. 16, 2023

Related U.S. Application Data
(60) Provisional application No. 63/399,582, filed on Aug. 19, 2022.

(51) Int. Cl. A61B 5/11; A61B 5/00

(52) U.S. Cl. CPC A61B 5/1126; A61B 5/681; G02B 6/4298

(57) ABSTRACT

An optical sensor includes a waveguide for directing, receiving, and coherently mixing electromagnetic radiation from an electromagnetic radiation source to detect one or more physical phenomena. The waveguide is integrable into a printed circuit board, allowing the optical sensor to maintain a small footprint.

### CROSS-REFERENCE TO RELATED APPLICATION

This application is a nonprovisional and claims the benefit under 35 U.S.C. § 119(e) of U.S. Provisional Patent Application No. 63/399,582, filed Aug. 19, 2022, the contents of which are incorporated herein by reference as if fully disclosed herein.

### TECHNICAL FIELD

Embodiments described herein relate to optical sensors, and in particular to waveguides for use with optical sensors.

### BACKGROUND

Optical sensors may sense physical phenomena such as movement, environmental conditions, and biometric data about a user. Accordingly, wearable electronic devices such as smart watches may incorporate optical sensors to sense data about the user thereof and/or the surrounding environment. Given the wide range of applications for optical sensors, any new development in the configuration or operation of the sensors can be useful. New developments that may be particularly useful are developments that provide additional sensing capability while maintaining a small form factor.

### SUMMARY

Embodiments described herein relate to optical sensors including a waveguide for directing, receiving, and coherently mixing electromagnetic radiation from an electromagnetic radiation source in order to detect one or more physical phenomena. In a first aspect, a waveguide for an optical sensor includes an input, a splitter coupled to the input, a subject output aperture coupled to the splitter, a reference coupler coupled to the splitter, a subject input aperture, a combiner coupled to the reference coupler and the subject input aperture, a mixer coupled to the combiner, a measuring aperture coupled to the mixer, and a printed circuit board (PCB) on which the input, the splitter, the subject output aperture, the reference coupler, the subject input aperture, the combiner, the mixer, and the measurement aperture are disposed. The input is configured to receive electromagnetic radiation from an electromagnetic radiation source. The splitter is configured to split the electromagnetic radiation from the electromagnetic radiation source into a measuring portion of electromagnetic radiation and a reference portion of electromagnetic radiation. The measuring portion of electromagnetic radiation is directed towards a subject through the subject output aperture. The reference coupler is configured to receive the reference portion of electromagnetic radiation. The subject input aperture is configured to receive reflections of the measuring portion of electromagnetic radiation from the subject. The combiner is configured to combine the reference portion of electromagnetic radiation and the reflections of the measuring portion of electromagnetic radiation from the subject. The mixer is configured to coherently mix the reference portion of electromagnetic radiation and the reflections of the measuring portion of electromagnetic radiation to provide a mixed signal. The mixed signal is provided to an electromagnetic radiation sensor through the measuring aperture.

The waveguide may further include a subject output reflector, a subject input reflector, and a measuring reflector. The subject output reflector may be configured to direct the measuring portion of electromagnetic radiation from the splitter through the subject output aperture at a non-parallel angle to a direction of propagation of the waveguide. The subject input reflector may be configured to direct the reflections of the measuring portion of electromagnetic radiation from the subject input aperture to the combiner along the direction of propagation of the waveguide. The measuring reflector may be configured to direct the mixed signal from the mixer through the measuring aperture at a non-parallel angle to the direction of propagation of the waveguide.

The subject output aperture, the subject input aperture, and the measurement aperture may extend through at least a portion of the PCB. The waveguide may comprise plastic. The input, the splitter, the subject output aperture, the reference coupler, the subject input aperture, the combiner, the mixer, and the measuring aperture may be a monolithic structure.

The mixer may be configured to coherently mix the reference portion of electromagnetic radiation and the reflections of the measurement portion of electromagnetic radiation to isolate a doppler shift in the measurement portion of the electromagnetic radiation caused by movement of the subject and/or movement within the subject.

The mixer may be configured to coherently mix the reference portion of electromagnetic radiation and the reflections of the measurement portion of electromagnetic radiation to isolate one or more modes in the reflections of the measurement portion of electromagnetic radiation.

In various aspects, the waveguide may be a multimode waveguide or a single mode waveguide.

In one aspect, an optical sensor includes an electromagnetic radiation source, an electromagnetic radiation sensor, a waveguide coupled to the electromagnetic radiation source and the electromagnetic radiation sensor, and a PCB on which the electromagnetic radiation source, the electromagnetic radiation sensor, and the waveguide are disposed. The waveguide may include an input, a splitter coupled to the input, a subject output aperture coupled to the splitter, a reference coupler coupled to the splitter, a subject input aperture, a combiner coupled to the reference coupler and the subject input aperture, a mixer coupled to the combiner, and a measuring aperture coupled to the mixer. The input is configured to receive electromagnetic radiation from the electromagnetic radiation source. The splitter is configured to split the electromagnetic radiation from the electromagnetic radiation source into a measuring portion of electromagnetic radiation and a reference portion of electromagnetic radiation. The measuring portion of electromagnetic radiation is directed towards a subject through the subject output aperture. The reference coupler is configured to receive the reference portion of electromagnetic radiation. The subject input aperture is configured to receive reflections of the measuring portion of electromagnetic radiation from the subject. The combiner is configured to combine the reference portion of electromagnetic radiation and the reflections of the measuring portion of electromagnetic radiation from the subject. The mixer is configured to coherently mix the reference portion of electromagnetic radiation and the reflections of the measuring portion of electromagnetic radiation to provide a mixed signal. The mixed signal is provided to the electromagnetic radiation sensor through the measuring aperture.

The waveguide may further include a subject output reflector, a subject input reflector, and a measuring reflector. The subject output reflector may be configured to direct the measuring portion of electromagnetic radiation from the splitter through the subject output aperture at a non-parallel angle to a direction of propagation of the waveguide. The subject input reflector may be configured to direct the reflections of the measuring portion of electromagnetic radiation from the subject input aperture to the combiner along the direction of propagation of the waveguide. The measuring reflector may be configured to direct the mixed signal from the mixer through the measuring aperture at a non-parallel angle to the direction of propagation of the waveguide.

The subject output aperture, the subject input aperture, and the measurement aperture may extend through at least a portion of the PCB. The waveguide may comprise plastic. The input, the splitter, the subject output aperture, the reference coupler, the subject input aperture, the combiner, the mixer, and the measuring aperture may be a monolithic structure.

The mixer may be configured to coherently mix the reference portion of electromagnetic radiation and the reflections of the measurement portion of electromagnetic radiation to isolate a doppler shift in the measurement of the electromagnetic radiation caused by movement of the subject and/or movement within the subject.

The mixer may be configured to coherently mix the reference portion of electromagnetic radiation and the reflections of the measurement portion of electromagnetic radiation to isolate one or more modes in the reflections of the measurement portion of electromagnetic radiation.

In various aspects, the waveguide may be a multimode waveguide or a single mode waveguide.

Embodiments are also direct to an optical sensor that includes an electromagnetic radiation source, an electromagnetic radiation sensor, and a light pipe coupled to the electromagnetic radiation source and the electromagnetic radiation sensor. The light pipe can include an input configured to receive electromagnetic radiation from an electromagnetic radiation source, a subject output reflector configured to direct the measurement portion of electromagnetic radiation from the splitter through the subject output aperture at a non-parallel angle to a direction of propagation of the waveguide and a subject input reflector configured to direct the reflections of the measuring portion of electromagnetic radiation from the subject input aperture to the combiner along the direction of propagation of the waveguide. The light pipe can also include a combiner coupled to the reference coupler and the subject input aperture and configured to combine the reference portion of electromagnetic radiation and the reflections of the measuring portion of electromagnetic radiation from the subject and a mixer coupled to the combiner and configured to coherently mix the reference portion of electromagnetic radiation and the reflections of the measuring portion of electromagnetic radiation from the subject to provide a mixed signal. The light pipe can include a measuring reflector configured to direct the mixed signal from the mixer through the measuring aperture at a non-parallel angle to the direction of propagation of the waveguide.

### BRIEF DESCRIPTION OF THE DRAWINGS

Reference will now be made to representative embodiments illustrated in the accompanying figures. It should be understood that the following descriptions are not intended to limit this disclosure to one included embodiment. To the contrary, the disclosure provided herein is intended to cover alternatives, modifications, and equivalents as may be included within the spirit and scope of the described embodiments, and as defined by the appended claims.

FIG. 1 is a functional block diagram illustrating an optical sensor, such as described herein.

FIGS. 2A and 2B show a cross-sectional view and a top view, respectively, illustrating an optical sensor, such as described herein.

FIG. 3 is a graph illustrating example splitting ratio and power ratio data for a multimode waveguide, such as described herein.

FIG. 4 shows a cross-sectional view of an example optical sensor, as described herein.

FIGS. 5A and 5B show an isometric front view and an isometric back view of a wearable device, such as described herein.

FIG. 6 is an electrical block diagram illustrating a device, such as described herein.

The use of the same or similar reference numerals in different figures indicates similar, related, or identical items.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Similarly, certain accompanying figures include vectors, rays, traces and/or other visual representations of one or more example paths—which may include reflections, refractions, diffractions, and so on, through one or more mediums—that may be taken by, or may be presented to represent, one or more photons, wavelets, or other propagating electromagnetic energy originating from, or generated by, one or more light sources shown or, or in some cases, omitted from, the accompanying figures. It is understood that these simplified visual representations of light or, more generally, electromagnetic energy, regardless of spectrum (e.g., ultraviolet, visible light, infrared, and so on), are provided merely to facilitate an understanding of the various embodiments described herein and, accordingly, may not necessarily be presented or illustrated to scale or with angular precision or accuracy, and, as such, are not intended to indicate any preference or requirement for an illustrated embodiment to receive, emit, reflect, refract, focus, and/or diffract light at any particular illustrated angle, orientation, polarization, color, or direction, to the exclusion of other embodiments described or referenced herein.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Embodiments described herein relate to optical sensors including a waveguide for directing, receiving, and coherently mixing electromagnetic radiation from an electromagnetic radiation source in order to detect one or more physical phenomena. Optical sensors are being investigated for sensing an increasing array of physical phenomena. For example, optical measurement techniques such as self-mixing interferometry (SMI) and laser doppler interferometry (LDI) are being investigated for sensing biometric data about a user such as blood flow information and blood pressure information. As consumer demand for detailed biometric and health data continues to increase, the demand for wearable devices including these and other optical sensors similarly continues to rise. However, integrating optical sensors into a wearable device often proves challenging due to size and power constraints. Waveguides described herein, as well as optical sensors including these waveguides, may enable new optical sensing capabilities in a wearable device.

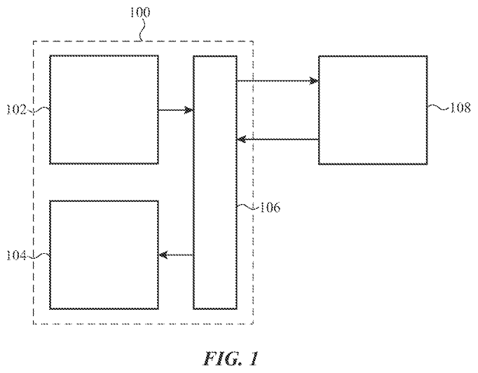

These foregoing and other embodiments are discussed below with reference to FIGS. 1-4. However, those skilled in the art will readily appreciate that the detailed description given herein with respect to these figures is for explanation only and should not be construed as limiting.

FIG. 1 is a diagram illustrating the basic components of an exemplary optical sensor 100 according to one aspect of the present disclosure. The optical sensor 100 may include an electromagnetic radiation source 102, an electromagnetic radiation sensor 104, and a waveguide 106 between the electromagnetic radiation source 102 and the electromagnetic radiation sensor 104. In operation, the electromagnetic radiation source 102 may generate and emit electromagnetic radiation. The electromagnetic radiation generated and emitted by the electromagnetic radiation source 102 may have one or more desired characteristics such as a desired intensity, wavelength, mode(s), etc. The electromagnetic radiation from the electromagnetic radiation source 102 may be coupled into the waveguide 106, where it is split into a measurement portion of electromagnetic radiation and a reference portion of electromagnetic radiation. The electromagnetic radiation may be split in any desired manner, such as, for example, 50% to the measurement portion of electromagnetic radiation and 50% to the reference portion of electromagnetic radiation. The measurement portion of electromagnetic radiation may be directed towards a subject 108, which, in various aspects may be one or more anatomical features of a person (e.g., the wrist of a user). Some of the measurement portion of electromagnetic radiation may be reflected from the user back towards the waveguide 106, where it is coherently mixed with the reference portion of electromagnetic radiation to provide a mixed signal. The mixed signal may be provided to and sensed by the electromagnetic radiation sensor 104, and may contain useful information about one or more physical phenomena associated with the subject 108, such as movement or biometric data.

In various aspects the waveguide 106 may be a single mode waveguide or a multimode waveguide. The waveguide 106 may be configured to coherently mix the reference portion of electromagnetic radiation and the reflections of the measurement portion of electromagnetic radiation to isolate a doppler shift in the measurement portion of electromagnetic radiation caused by movement of the subject 108 or by movement within the subject 108 (e.g., when the measurement portion of electromagnetic radiation penetrates a surface of the subject 108). In other aspects, the waveguide 106 may be configured to coherently mix the reference portion of electromagnetic radiation and the reflections of the measurement portion of electromagnetic radiation to isolate one or more modes in the reflections of the measurement portion of electromagnetic radiation. Even if the electromagnetic radiation generated and emitted by the electromagnetic radiation source 102 is provided having a single mode or a limited subset of modes, when the measurement portion of electromagnetic radiation interacts with the subject 108, the electromagnetic radiation scatters and reflects, creating many different modes in the reflections of the measurement portion of electromagnetic radiation received by the waveguide 106. The different modes in the reflections of the measurement portion of electromagnetic radiation may make it difficult to measure some physical phenomena. By coherently mixing the reflections of the measurement portion of electromagnetic radiation and the reference portion of electromagnetic radiation, the resulting mixed signal may enable the measurement of some physical phenomena. As will be discussed herein, the waveguide provides this functionality in a small footprint that may be suitable for wearable devices.

In various aspects, the electromagnetic radiation source 102 may be a coherent light source such as a laser. In particular, the electromagnetic radiation source 102 may be a distributed feedback laser (DFB), vertical cavity surface-emitting laser (VCSEL), an edge-emitting laser (EEL), a horizontal cavity surface-emitting laser (HCSEL), a vertical external-cavity surface-emitting laser (VECSEL), a quantum dot laser (QDL), a quantum cascade laser (QCL), or one or more light-emitting diodes (LEDs) such as organic LEDs (OLEDs), resonant cavity LEDs (RC-LEDs), micro-LEDs (mLEDs), superluminescent LEDs (SLEDs), and edge-emitting (ELEDs). The electromagnetic radiation sensor 104 may include one or more photodiodes, or any suitable sensor for sensing electromagnetic radiation. In some aspects, the electromagnetic radiation sensor 104 may be a balanced or differential sensor including at least two sensing parts configured to sense electromagnetic radiation from different parts of the waveguide. For example, the electromagnetic radiation sensor 104 may include at least two photosensors providing a differential output signal. Providing the electromagnetic radiation sensor 104 as a differential sensor may increase the sensitivity of the optical sensor 100 by reducing the noise floor thereof.

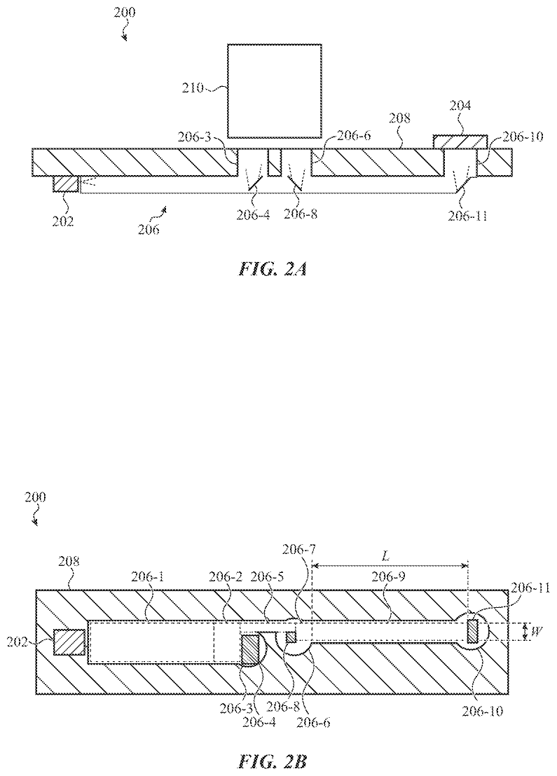

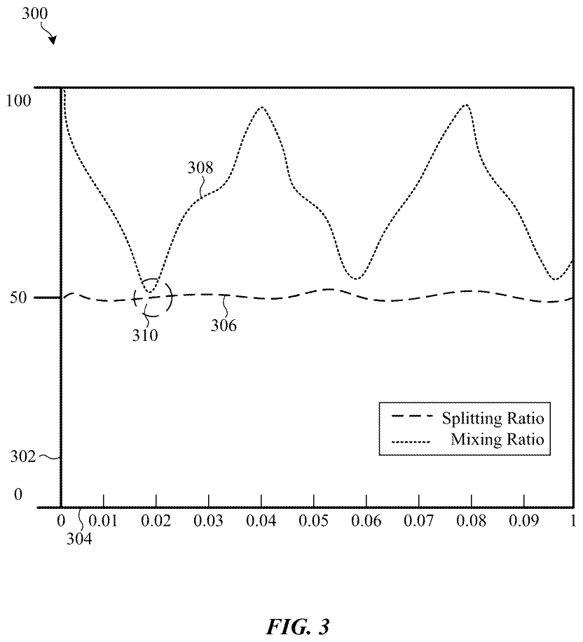

FIGS. 2A and 2B are diagrams illustrating a physical layout for an optical sensor 200 according to one aspect of the present disclosure. In particular, FIG. 2A shows a cross-sectional view of the optical sensor 200 while FIG. 2B shows a top-view of the optical sensor 200. The optical sensor 200 may include an electromagnetic radiation source 202, an electromagnetic radiation sensor 204, a waveguide 206 optically coupled between the electromagnetic radiation source 202 and the electromagnetic radiation sensor 204, and a PCB 208. The electromagnetic radiation source 202, the electromagnetic radiation sensor 204, and the waveguide 206 may be disposed on and/or integrated into the PCB 208.

In operation, the electromagnetic radiation source 202 may generate and emit electromagnetic radiation having one or more desired characteristics such as intensity, wavelength, mode(s), etc. As shown in FIG. 2B, the waveguide 206 may be divided into several functional sections, which in various aspects may be separate components or different parts of the same component. That is, the waveguide 206 may be a monolithic structure or include any number of discrete components. In particular, the waveguide 206 may include an input 206-1 configured to receive the electromagnetic radiation from the electromagnetic radiation source 202. A splitter 206-2 may be coupled to the input 206-1 and configured to split the electromagnetic radiation from the electromagnetic radiation source 202 into a measurement portion of electromagnetic radiation and a reference portion of electromagnetic radiation. The electromagnetic radiation may be split in any desired manner, such as, for example, 50% to the measurement portion of electromagnetic radiation and 50% to the reference portion of electromagnetic radiation. A subject output aperture 206-3 may be coupled to the splitter 206-2, and the measurement portion of electromagnetic radiation may be directed through the subject output aperture 206-3 via a subject output reflector 206-4 towards a subject 210. In particular, the subject output reflector 206-4 may direct the measurement portion of electromagnetic radiation through the subject output aperture 206-3 at a non-parallel angle to a direction of propagation of the waveguide 206 (e.g., perpendicular to the direction of propagation). A reference coupler 206-5 may be coupled to the splitter 206-2 and configured to receive the reference portion of electromagnetic radiation. A subject input aperture 206-6 may be configured to receive reflections of the measurement portion of electromagnetic radiation from the subject 210. A combiner 206-7 may be coupled to the reference coupler 206-5 and the subject input aperture 206-6 and configured to combine the reference portion of electromagnetic radiation and reflections of the measuring portion of electromagnetic radiation. A subject input reflector 206-8 may be configured to direct the reflections of the measuring portion of electromagnetic radiation into the combiner 206-7. In particular, the subject input reflector 206-8 may be configured to direct the reflections of the measuring portion of electromagnetic radiation into the combiner 206-7 along the direction of propagation of the waveguide 206. A mixer 206-9 may be coupled to the combiner 206-7 and configured to coherently mix the reference portion of electromagnetic radiation and the reflections of the measuring portion of electromagnetic radiation to provide a mixed signal. A measuring aperture 206-10 may be coupled between the mixer 206-9 and the electromagnetic radiation sensor 204, and the mixed signal may be provided to the electromagnetic radiation sensor 204 via the measuring aperture 206-10. A measuring reflector 206-11 may be configured to direct the mixed signal from the mixer 206-9 through the measuring aperture 206-10. In particular, the measuring reflector 206-11 may direct the mixed signal from the mixer 206-9 through the measuring aperture 206-10 at a non-parallel angle to the direction of propagation of the waveguide 206 (e.g., perpendicular to the direction of propagation). The mixed signal may be sensed by the electromagnetic radiation sensor 204, and may include useful information about one or more physical phenomena associated with the subject 210, such as movement or biometric data.

While the electromagnetic radiation sensor 204 is shown on an opposite side of the PCB 208 as the electromagnetic radiation source 202, in some aspects the electromagnetic radiation sensor 204 may be provided on the same side of the PCB 208 as the electromagnetic radiation source 202. In such an embodiment, the measuring reflector 206-11 may be omitted.

In various aspects the waveguide 206 may be a single mode waveguide or a multimode waveguide. The waveguide 206 may be configured to coherently mix the reference portion of electromagnetic radiation and the reflections of the measurement portion of electromagnetic radiation to isolate a doppler shift in the measurement portion of electromagnetic radiation caused by movement of the subject 210 and/or movement within the subject 210 (e.g., when the measurement portion of electromagnetic radiation penetrates a surface of the subject 210). In other aspects, the waveguide 206 may be configured to coherently mix the reference portion of electromagnetic radiation and the reflections of the measurement portion of electromagnetic radiation to isolate a mode in the reflections of the measurement portion of electromagnetic radiation. Even if the electromagnetic radiation generated and emitted by the electromagnetic radiation source 202 is provided having a single mode or limited subset of modes, when the measurement portion of electromagnetic radiation interacts with the subject 210, the electromagnetic radiation scatters and reflects, creating many different modes in the reflections of the measurement portion of electromagnetic radiation received by the waveguide 206. The different modes in the reflections of the measurement portion of electromagnetic radiation may make it difficult to measure some physical phenomena. By coherently mixing the reflections of the measurement portion of electromagnetic radiation and the reference portion of electromagnetic radiation, the resulting mixed signal may enable the measurement of some physical phenomena.

To achieve coherent mixing of the reflections of the measurement portion of electromagnetic radiation and the reference portion of electromagnetic radiation, the dimensions of the mixer 206-9 may be carefully designed. Those skilled in the art will appreciate that a length L of the mixer 206-9 and a width W of the mixer 206-9 may affect the interaction between the modes of the reflections of the measurement portion of electromagnetic radiation and the reference portion of electromagnetic radiation. In some aspects, the length L and width W of the mixer 206-9 may be designed to achieve a 50%±10% power ratio at the surface of the mixer 206-9 coupled to the measuring aperture 206-10 and a 50%±10% mode mixing ratio. While not shown, the waveguide 206 may further split the electromagnetic radiation generated by the electromagnetic radiation source 202 into additional measurement and reference portions, which are directed to additional apertures and mixers that operate in a similar manner as described above for measuring physical phenomena at various physical locations on the subject 210.

In addition to the length L and width W of the mixer 206-9, a relationship between a coherence length of the electromagnetic radiation source 202, a length of the reference coupler 206-5, and the dimensions of the mixer 206-9 may be adjusted to set a desired sensing depth for the optical sensor 200. Those skilled in the art will appreciate that a distance between the electromagnetic radiation source 202 and the electromagnetic radiation sensor 204, along with the dimensions of the various parts of the waveguide 206, may determine a sensing depth of the optical sensor 200. Accordingly, these and any other parameters may be adjusted to achieve a desired sensing depth (e.g., a depth at which blood vessels are often located).

Providing the waveguide 206 as a multimode waveguide may provide advantages in manufacturing of the optical sensor 200. In particular, providing the waveguide 206 as a multimode waveguide may ease tolerances associated with the relative positioning between the electromagnetic radiation source 202 and the waveguide 206 that would otherwise be much smaller if the waveguide 206 was a single mode waveguide. If the reflection surface of the waveguide 206 reflects a wide variety of modes, the noise caused by additional modes may be drowned out by amplification caused by the reference portion of electromagnetic radiation, thereby allowing for the advantage of eased manufacturing tolerances while still enabling a high signal to noise ratio (SNR).

FIG. 3 is an example graph 300 showing splitting ratio 306 and mixing ratio 308 for a multimode waveguide, such as waveguides 200 and 400 described herein. The vertical axis 302 shows the normalized ratio between a reference portion of electromagnetic radiation and a measurement portion of electromagnetic radiation and the horizontal axis 304 represents a length of the waveguide (e.g., length L of the mixer 206-9). In some cases, multimode waveguides with smaller numerical apertures, may cause one or more modes to periodically reconstruct, which may increase a mixing of the multimode measurement electromagnetic radiation with the reference electromagnetic radiation. Point 310 shows an example of the multimode waves reconstructing to achieve a mixing ratio at about 50%. As discussed above, a optical sensor may be configured to achieve reconstruction of light by using a multimode waveguide and this may reduce manufacturing and/or alignment tolerance of the optical sensor (e.g., optical sensors 200 and 400).

The PCB 208 may include one or more layers of substrate, one or more conductive layers, and one or more finishing layers (e.g., solder masks and silkscreens). The substrate may include any suitable material for supporting conductive traces and components, such as epoxy laminates and polyimide films. As shown, the waveguide 206 may be disposed on and integrated into the PCB 208. In various aspects, the waveguide 206 may be disposed on a first surface of the PCB 208, and the subject output aperture 206-3, the subject input aperture 206-6, and the measuring aperture 206-10 may extend through the PCB 208 to a second surface of the PCB 208 opposite the first surface such that the waveguide 206 directs light through the PCB 208 (from one side to another). In various aspects, the waveguide 206 may comprise a polymer such as plastic. However, the waveguide 206 may comprise any material that is readily integrable with a PCB. The waveguide 206 may be a monolithic structure provided by a single piece of material. That is, the input 206-1, the splitter 206-2, the subject output aperture 206-3, the reference coupler 206-5, the subject input aperture 206-6, the combiner 206-7, the mixer 206-9, and the subject output aperture 206-10 may be part of the same piece of material (e.g., the same piece of plastic). However, the waveguide 206 may also be formed by one or more discrete components forming some or all of the functional parts described herein. The waveguide 206 may be readily integrated at any layer within the PCB 208, and in some aspects may be located on an internal layer of the PCB 208 (i.e., the waveguide 206 may be covered on both sides by the PCB 208 or completely encapsulated by the PCB 208, aside from where the subject output aperture 206-3, the subject input aperture 206-6, and the measuring aperture 206-10 are exposed). When the waveguide 206 is located on an interior layer of the PCB 208, the subject output aperture 206-3, the subject input aperture 206-6, and the measuring aperture 206-10 may extend through only a portion of the PCB 208. Integrating waveguide 206 into an interior layer of the PCB 208 may offer significant size reductions in a device incorporating the waveguide 206 and PCB 208, as it may allow for electrical components to be placed above and/or below the waveguide 206 on an exposed surface of the PCB 208. Accordingly, the waveguide 206 may be added to one or more existing PCBs in a wearable or other device with minimal impact on the footprint thereof. While the waveguide 206 is primarily discussed as being integrable with a PCB, those skilled in the art will readily appreciate that the principles of the present disclosure may similarly apply to integrating a waveguide 206 into any type of substrate or material.

The subject output reflector 206-4, the subject input reflector 206-8, and the measuring reflector 206-11 may be provided by any suitable process or materials, such as via a grayscale lithography process performed on a surface of the waveguide 206, by cutting (to a desired angle) and polishing a surface of the waveguide 206, or by placing a reflective material such as gold on a surface of the waveguide 206. In general, the subject output reflector 206-4, the subject input reflector 206-8, and the measuring reflector 206-11 may be discrete components, or may be provided as coatings, surface treatments (e.g., polishing), and the like.

In various aspects, the electromagnetic radiation source 202 may be a coherent light source such as a laser. In particular, the electromagnetic radiation source 202 may be a DFB, a VCSEL, an EEL, a HCSEL, a VECSEL, a QDL, a QCL, or one or more LEDs such as organic OLEDs, RC-LEDs, mLEDs, SLEDs, and edge-emitting LEDs. The electromagnetic radiation sensor 204 may include one or more photodetectors such as photodiodes, or any suitable sensor for measuring electromagnetic radiation. In some aspects, the electromagnetic radiation sensor 204 may be a balanced or differential sensor including at least two sensing parts configured to sense electromagnetic radiation from different of the waveguide 206. For example, the electromagnetic radiation sensor 204 may include at least two photodetectors providing a differential output signal. Providing the electromagnetic radiation sensor 204 as a differential sensor may increase the sensitivity of the optical sensor 200 by reducing the noise floor thereof.

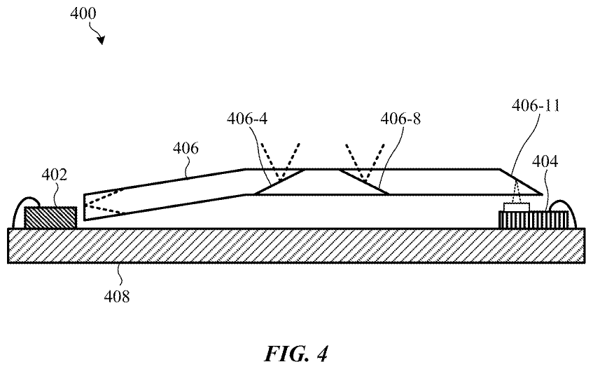

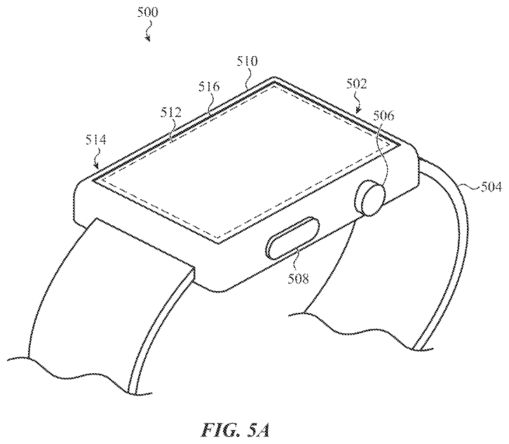

FIG. 4 shows a cross-sectional view of an example optical sensor 400, as described herein. The optical sensor 400 may include a waveguide 406 that is formed from a light pipe. The optical sensor 200 may include an electromagnetic radiation source 402, an electromagnetic radiation sensor 404, the light pipe 406 optically coupled between the electromagnetic radiation source 402 and the electromagnetic radiation sensor 404, and a base 408. The base may be any suitable substrate which may be separate from or integrated with a PCB. The optical sensor 400 may be an example of the optical sensors described herein (e.g., optical sensor 200).

The optical sensor 400 may include a single mode or multimode light pipe and function to coherently mix the reference portion of electromagnetic radiation and the reflections of the measurement portion of electromagnetic radiation to isolate a doppler shift in the measurement portion of electromagnetic radiation caused by movement of a subject or by movement within the subject (e.g., when the measurement portion of electromagnetic radiation penetrates a surface of the subject 108), as described herein. In other aspects, the optical sensor 400 may be configured to coherently mix the reference portion of electromagnetic radiation and the reflections of the measurement portion of electromagnetic radiation to isolate one or more modes in the reflections of the measurement portion of electromagnetic radiation, as described herein. By coherently mixing the reflections of the measurement portion of electromagnetic radiation and the reference portion of electromagnetic radiation, the resulting mixed signal may enable the measurement of some physical phenomena.

In operation, the electromagnetic radiation source 402 may generate and emit electromagnetic radiation having one or more desired characteristics such as intensity, wavelength, mode(s), etc. The light pipe 406 may include a subject output 406-4 that directs a portion of the emitted electromagnetic radiation towards a subject. In particular, the subject output 406-4 may direct a measurement portion of electromagnetic radiation at a non-parallel angle to a direction of propagation of the light pipe 406 (e.g., perpendicular to the direction of propagation). The subject output 406-4 may include a reflector, photodetectors attached to the waveguide/light pipe, and/or have the waveguide extend to an edge of the optical sensor. The light pipe 406 may include a subject input 406-8, which may be configured to receive reflections of the measuring portion of electromagnetic radiation from a user and direct the reflections back into the light pipe 406. The subject input 406-8 may include a reflector, photodetectors attached to the waveguide/light pipe, and/or have the waveguide extend to an edge of the optical sensor. The measurement portions of the electromagnetic radiation may be combined with the reference portion of electromagnetic radiation, as described herein (e.g., using a combiner as described herein). In some cases, the measurement portions (e.g., a measuring reflector) may include a reflector, photodetectors attached to the waveguide/light pipe, and/or have the waveguide extend to an edge of the optical sensor.

The light pipe 406 may coherently mix the reference portion of electromagnetic radiation and the reflections of the measuring portion of electromagnetic radiation to provide a mixed signal (e.g., using a mixer as described herein). A measuring reflector 406-11 may be configured to direct the mixed signal to the radiation sensor 404. The mixed signal may be sensed by the electromagnetic radiation sensor 404, and may include useful information about one or more physical phenomena associated with the subject, such as movement or biometric data.

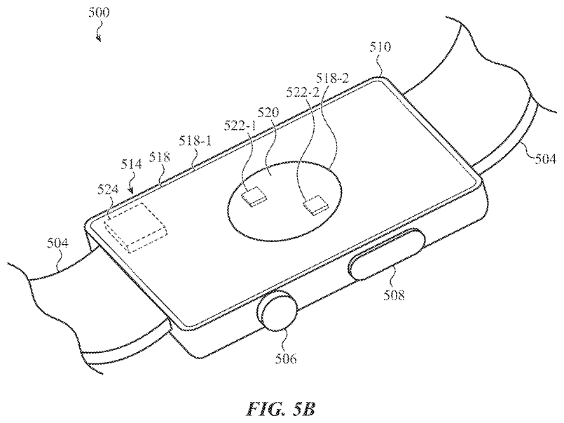

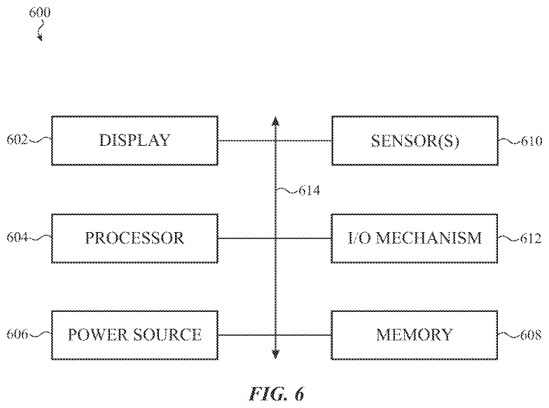

FIGS. 5A and 5B show an example of a wearable device 500 that may incorporate one or more sensors, including one or more optical sensors as discussed herein. Specifically, FIG. 5A shows a front isometric view of the wearable device 500, while FIG. 5B shows a back isometric view of the wearable device 500. The sensors of the wearable device 500 may be used, for example, to acquire biometric data from a user (e.g., heart rate, respiration rate, blood pressure, blood flow rate, blood oxygenation, blood glucose level), or to determine a status of the wearable device 500 (e.g., whether the wearable device 500 is being worn, a tightness of the wearable device 500 as secured to the user, one or more ambient environmental conditions). While the wearable device 500 is illustrated having the form factor of a watch, the wearable device 500 could be any suitable type of wearable device having any form factor. Further, the principles described herein equally apply to non-wearable devices such as smartphones, tablets, laptop computers, desktop computers, and the like.

The wearable device 500 includes a body 502 (e.g., a watch body) and a band 504. The body 502 may include an input or selection device, such as a crown 506 or a button 508. The band 504 may be attached to a housing 510 of the body 502, and may be used to attach the body 502 to a body part of a user (e.g., an arm, wrist, leg, ankle, or waist). The housing 510 may at least partially surround a display 512. In some embodiments, the housing 510 may include a sidewall 514, which may support a front cover 516 (shown in FIG. 5A) and/or a back cover 518 (shown in FIG. 5B). The front cover 516 may be positioned over the display 512, and may provide a window through which the display 512 is viewed. In some aspects, the display 512 may be attached to (or about) the sidewall 514 and/or the front cover 516. In other aspects, the display 512 may not be included and/or the housing 510 may have an alternative configuration.

The display 512 may include one or more light-emitting elements including, for example, light-emitting elements that define an LED display, an OLED display, a liquid crystal display (LCD), electroluminescent (EL) display, or any other type of display. In some aspects, the display 512 may include, or be associated with, one or more touch and/or force sensors that are configured to detect a touch and/or force applied to a surface of the front cover 516.

In some aspects, the sidewall 514 of the housing 510 may be formed using one or more metals (e.g., aluminum or stainless steel), polymers (e.g., plastics), ceramics, or composites (e.g., carbon fiber). The front cover 516 may be formed, for example, using one or more of glass, a crystal (e.g., sapphire), or a transparent polymer (e.g., plastic) that enables a user to view the display 512 through the front cover 516. In some cases, a portion of the front cover 516 (e.g., a perimeter of the front cover 516) may be coated with an opaque ink to obscure components included within the housing 510. In some cases, all of the exterior components of the housing 510 may be formed from a transparent material, and components within the wearable device 500 may or may not be obscured by an opaque ink or opaque structure within the housing 510.

The back cover 518 may be formed using the same material or materials used to form the sidewall 514 and/or the front cover 516. In some cases, the back cover 518 may be part of a monolithic element that also forms the sidewall 514. In other cases, and as shown, the back cover 518 may be a multi-part back cover, such as a back cover having a first back cover 518-1 attached to the sidewall 514 and a second back cover 518-2 attached to the first back cover 518-1. The second back cover 518-2 may in some aspects have a circular perimeter and an arcuate exterior surface 520 (i.e., an exterior surface 520 having an arcuate profile).

The front cover 516, the back cover 518, and the first back cover 518-1 may be mounted to the sidewall 514 using fasteners, adhesives, seals, gaskets, or other components. The second back cover 518-2, when present, may be mounted to the first back cover 518-1 using fasteners, adhesives, seals, gaskets, or other components.

A display stack or device stack (hereinafter referred to as a “stack”) including the display 512 may be attached (or abutted) to an interior surface of the front cover 516 and extend into an interior volume of the wearable device 500. In some cases, the stack may include a touch sensor (e.g., a grid of capacitive, resistive, strain-based, ultrasonic, or other type of touch sensing elements), or other layers of optical, mechanical, electrical, or other types of components. In some cases, the touch sensor (or part of a touch sensor system) may be configured to detect a touch applied to an outer surface of the front cover 516 (e.g., to a display surface of the wearable device 500).

The wearable device 500 may include various sensors 522. For purposes of illustration, the wearable device 500 is shown including a first sensor 522-1 and a second sensor 522-2. The first sensor 522-1 may be an optical sensor including a waveguide as discussed herein, and may be used to sense various biometric data about the user including heart rate, respiration rate, blood oxygenation, blood flow information, blood pressure, etc. The second sensor 522-2 may be a different type of sensor such as a photoplethysmography (PPG) sensor, which may be used to sense the same or different biometric data about the user. As discussed herein, the waveguide of the first sensor 522-1 may enable the integration thereof into the wearable device 500 with a minimal footprint, and may enable the sensing of previously unavailable biometric data, or may increase the accuracy of biometric data sensed using other sensors such as the second sensor 522-2.

The wearable device 500 may include circuitry 524 (e.g., processing circuitry and/or other components) configured to determine or extract, at least partly in response to signals received directly or indirectly from sensors therein (e.g., the first sensor 522-1 and the second sensor 522-2), biometric data about the user and/or a status of the wearable device 500. In doing so, the circuitry 524 may process signals from sensors therein using any suitable transformations, approximations, mathematical operations, and/or machine learning models. In some aspects, the circuitry 524 may be configured to convey the determined or extracted parameters or statuses to the user of the wearable device 500. For example, the circuitry 524 may cause the indication or indications to be displayed on the display 512, indicated via audio or haptic outputs, transmitted via a wireless communications interface or other communications interface, and so on. The circuitry 524 may also or alternatively maintain or alter one or more settings, functions, or aspects of the wearable device 500, including, in some cases, what is displayed on the display 512.

To illustrate a more general functional device that may include one or more optical sensors including a waveguide as discussed herein, FIG. 6 shows a sample electrical block diagram of a device 600. The device 600 may include a display 602 (e.g., a light-emitting display), a processor 604 (also referred to herein as processing circuitry), a power source 606, a memory 608, or storage device, a sensor system 610, an input/output (I/O) mechanism 612 (e.g., an input/output device, input/output port, or haptic input/output interface). The processor 604 may communicate, either directly or indirectly, with some or all of the other components of the device 600. For example, a system bus or other communication mechanism 614 can provide communication between the display 602, the processor 604, the power source 606, the memory 608, the sensor system 610, and the I/O mechanism 612.

The processor 604 may be implemented as any electronic device capable of processing, receiving, or transmitting data or instructions, whether such data or instructions is in the form of software or firmware or otherwise encoded. For example, the processor 604 may include a microprocessor, central processing unit (CPU), an application-specific integrated circuit (ASIC), a digital signal processor (DSP), a controller, or a combination of such devices. As described herein, the term “processor” or “processing circuitry” is meant to encompass a single processing unit, multiple processors, multiple processing units, or other suitably configured computing element or elements. In some aspects, the processor 604 may provide part or all of the processing systems, processing circuitry, or processors described with reference to any of FIGS. 1-5B.

It should be noted that the components of the device 600 can be controlled by multiple processors. For example, select components of the device 600 (e.g., the sensor system 610) may be controlled by a first processor and other components of the wearable device (e.g., the display 602) may be controlled by a second processor, where the first and second processors may or may not be in communication with each other.

The power source 606 can be implemented with any device capable of providing energy to the device 600. For example, the power source 606 may include one or more batteries or rechargeable batteries. Additionally or alternatively, the power source 606 may include a power connector or power cord that connects the device 600 to another power source, such as a wall outlet.

The memory 608 may store electronic data that can be used by the device 600. For example, the memory 608 may store electrical data or content such as, for example, audio and video files, documents and applications, device settings and user preferences, timing signals, control signals, and data structures and databases. The memory 608 may include any type of memory. By way of example only, the memory 608 may include random access memory (RAM), read-only memory (ROM), flash memory, removeable memory, other types of storage elements, or combinations of such memory types.

The device 600 may also include one or more sensor systems 610 positioned almost anywhere thereon. For example, the sensor system 610 may include one or more optical sensors having a waveguide as discussed in FIGS. 1 through 4. The sensor system 610 may be configured to sense one or more types of parameters, such as but not limited to: vibration, light, touch, force, heat, movement, relative motion, biometric data (e.g., biological parameters) of a user, air quality, proximity, position, or connectedness. By way of example, the sensor system 610 may include one or more optical sensors including a waveguide as discussed in FIGS. 1 through 2B, a heat sensor, a position sensor, a light or optical sensor, an accelerometer, a pressure transducer, a gyroscope, a magnetometer, a health monitoring sensor, and/or an air quality sensor. Additionally, the one or more sensor systems 610 may utilize any suitable sensing technology including, but not limited to, interferometric, magnetic, capacitive, ultrasonic, resistive, optical, acoustic, piezoelectric, or thermal technologies.

The I/O mechanism 612 may transmit or receive data from a user or another electronic device. The I/O mechanism 612 may include the display 602, a touch sensing input surface, a crown, one or more buttons (e.g., a graphical user interface “home” button), one or more cameras (including an under-display camera), one or more microphones or speakers, one or more ports such as a microphone port, and/or a keyboard. Additionally or alternatively, the I/O mechanism 612 may transmit electronic signals via a communications interface, such as a wireless, wired, and/or optical communications interface. Examples of wireless and wired communications interfaces including, but are not limited to, cellular and Wi-Fi communications interfaces.

These foregoing embodiments depicted in FIGS. 1-6 and the various alternatives thereof and variations thereto are presented, generally, for purposes of explanation, and to facilitate an understanding of various configurations and constructions of a system, such as described herein. However, it will be apparent to one skilled in the art that some of the specific details presented herein may not be required in order to practice a particular described embodiment, or an equivalent thereof.

Thus, it is understood that the foregoing and following descriptions of specific embodiments are presented for the limited purposes of illustration and description. These descriptions are not targeted to be exhaustive or to limit the disclosure to the precise forms recited herein. To the contrary, it will be apparent to one of ordinary skill in the art that many modifications and variations are possible in view of the above teachings.

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

Although the disclosure above is described in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more other embodiments, whether or not such embodiments are described and whether or not such features are presented as being a part of a described embodiment. Thus, the breadth and scope of the present description should not be limited by any of the above-described exemplary embodiments but is instead defined by the claims herein presented.

The present disclosure recognizes that personal information data, including biometric data, in the present technology, can be used to the benefit of users. For example, the use of biometric authentication data can be used for convenient access to device features without the use of passwords. In other examples, user biometric data is collected for providing users with feedback about their health or fitness levels. Further, other uses for personal information data, including biometric data, that benefit the user are also contemplated by the present disclosure.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure, including the use of data encryption and security methods that meets or exceeds industry or government standards. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates embodiments in which users selectively block the use of, or access to, personal information data, including biometric data. That is, the present disclosure contemplates that hardware and/or software elements can be provided to prevent or block access to such personal information data. For example, in the case of biometric authentication methods, the present technology can be configured to allow users to optionally bypass biometric authentication steps by providing secure information such as passwords, personal identification numbers (PINS), touch gestures, or other authentication methods, alone or in combination, known to those of skill in the art. In another example, users can select to remove, disable, or restrict access to certain health-related applications collecting users' personal health or fitness data.

What is claimed is:

1. A waveguide for an optical sensor, comprising:
an input configured to receive electromagnetic radiation from an electromagnetic radiation source;
a splitter coupled to the input and configured to split the electromagnetic radiation from the electromagnetic radiation source into a measuring portion of electromagnetic radiation and a reference portion of electromagnetic radiation;
a subject output aperture coupled to the splitter, wherein the measuring portion of electromagnetic radiation is directed towards a subject through the subject output aperture;
a reference coupler coupled to the splitter and configured to receive the reference portion of electromagnetic radiation;
a subject input aperture configured to receive reflections of the measuring portion of electromagnetic radiation from the subject;
a combiner coupled to the reference coupler and the subject input aperture and configured to combine the reference portion of electromagnetic radiation and the reflections of the measuring portion of electromagnetic radiation from the subject;
a mixer coupled to the combiner and configured to coherently mix the reference portion of electromagnetic radiation and the reflections of the measuring portion of electromagnetic radiation from the subject to provide a mixed signal; and
a measuring aperture coupled to the mixer, wherein the mixed signal is provided to an electromagnetic radiation sensor through the measuring aperture.

2. The waveguide of claim 1, further comprising:
a subject output reflector configured to direct the measuring portion of electromagnetic radiation from the splitter through the subject output aperture at a non-parallel angle to a direction of propagation of the waveguide;
a subject input reflector configured to direct the reflections of the measuring portion of electromagnetic radiation from the subject input aperture to the combiner along the direction of propagation of the waveguide; and
a measuring reflector configured to direct the mixed signal from the mixer through the measuring aperture at a non-parallel angle to the direction of propagation of the waveguide.

3. The waveguide of claim 1, further comprising a printed circuit board (PCB) wherein:
the input, the splitter, the reference coupler, the combiner, and the mixer are disposed on the PCB; and
the subject output aperture, the subject input aperture, and the measuring aperture extend at least partially through the PCB.

4. The waveguide of claim 3, wherein the input, the splitter, the subject input aperture, the reference coupler, the subject input aperture, the combiner, the mixer, and the measuring aperture are a monolithic structure.

5. The waveguide of claim 1, wherein the mixer is configured to coherently mix the reference portion of electromagnetic radiation and the reflections of the measurement portion of electromagnetic radiation to isolate a doppler shift in the measurement of the electromagnetic radiation caused by movement of the subject.

6. The waveguide of claim 1, wherein the mixer is configured to coherently mix the reference portion of electromagnetic radiation and the reflections of the measurement portion of electromagnetic radiation to isolate one or more modes in the reflections of the measurement portion of electromagnetic radiation.

7. The waveguide of claim 1, wherein:
the waveguide is a multimode waveguide; and
the reflections of the measuring portion of electromagnetic radiation is scattered into multiple modes of the multimode waveguide.

8. The waveguide of claim 1, wherein the waveguide is a single mode waveguide.

9. An optical sensor, comprising:
an electromagnetic radiation source;
an electromagnetic radiation sensor; and
a waveguide coupled to the electromagnetic radiation source and the electromagnetic radiation sensor, the waveguide comprising:
an input configured to receive electromagnetic radiation from the electromagnetic radiation source;
a splitter coupled to the input and configured to split the electromagnetic radiation from the electromagnetic radiation source into a measuring portion of electromagnetic radiation and a reference portion of electromagnetic radiation;
a subject output aperture coupled to the splitter, wherein the measuring portion of electromagnetic radiation is directed towards a subject through the subject output aperture;
a reference coupler coupled to the splitter and configured to receive the reference portion of electromagnetic radiation;
a subject input aperture configured to receive reflections of the measuring portion of electromagnetic radiation from the subject;
a combiner coupled to the reference coupler and the subject input aperture and configured to combine the reference portion of electromagnetic radiation and the reflections of the measuring portion of electromagnetic radiation from the subject;
a mixer coupled to the combiner and configured to coherently mix the reference portion of electromagnetic radiation and the reflections of the measuring portion of electromagnetic radiation from the subject to provide a mixed signal; and
a measuring aperture coupled to the mixer, wherein the mixed signal is provided to the electromagnetic radiation sensor through the measuring aperture; and

10. The optical sensor of claim 9, wherein the waveguide further comprises:
a subject output reflector configured to direct the measurement portion of electromagnetic radiation from the splitter through the subject output aperture at a non-parallel angle to a direction of propagation of the waveguide;
a subject input reflector configured to direct the reflections of the measuring portion of electromagnetic radiation from the subject input aperture to the combiner along the direction of propagation of the waveguide; and
a measuring reflector configured to direct the mixed signal from the mixer through the measuring aperture at a non-parallel angle to the direction of propagation of the waveguide.

12. The optical sensor of claim 9, wherein the electromagnetic radiation source is a laser.

13. The optical sensor of claim 9, wherein the optical sensor comprises at least two photodetectors arranged so that a first portion of the mixed signal is provided to a first photodetector and a second portion of the mixed signal is provided to a second photodetector.

14. The optical sensor of claim 13, wherein an output signal of the optical sensor is a differential output signal from the at least two photodetectors.

15. The optical sensor of claim 10, wherein the waveguide is a multimode waveguide.

16. The optical sensor of claim 10, wherein the waveguide is a single mode waveguide.

17. An optical sensor, comprising:
an electromagnetic radiation source;
an electromagnetic radiation sensor; and
a light pipe coupled to the electromagnetic radiation source and the electromagnetic radiation sensor, the light pipe comprising:
an input configured to receive electromagnetic radiation from an electromagnetic radiation source;
a subject output configured to direct the measurement portion of electromagnetic radiation from the splitter through the subject output aperture;
a subject input configured to direct the reflections of the measuring portion of electromagnetic radiation from the subject input aperture to the combiner along the direction of propagation of the waveguide; and
a combiner coupled to the reference coupler and the subject input aperture and configured to combine the reference portion of electromagnetic radiation and the reflections of the measuring portion of electromagnetic radiation from the subject;
a mixer coupled to the combiner and configured to coherently mix the reference portion of electromagnetic radiation and the reflections of the measuring portion of electromagnetic radiation from the subject to provide a mixed signal; and
a waveguide configured to direct the mixed signal from the mixer through the measuring aperture.

18. The optical sensor of claim 17, wherein the light pipe is a multimode light pipe.

19. The optical sensor of claim 19, wherein the light pipe comprises a polymer material.

20. The optical sensor of claim 17, wherein the light pipe comprises a monolithic structure.
